SEQUENCE LISTING (9 sequences)

SEQ ID NO: 1 - protein, 7 aa, Drosophila melanogaster
AGAIAGG

SEQ ID NO: 2 - protein, 7 aa, Drosophila melanogaster
AIAGGVI

SEQ ID NO: 3 - protein, 7 aa, Drosophila melanogaster
AIAGGVV

SEQ ID NO: 4 - protein, 7 aa, Drosophila melanogaster
YYAGAGV

SEQ ID NO: 5 - protein, 7 aa, Drosophila melanogaster
AGAIAGG

SEQ ID NO: 6 - protein, 11 aa, Drosophila melanogaster
AGAIAGGVIGG

SEQ ID NO: 7 - protein, 4 aa, Drosophila melanogaster
ASGA

SEQ ID NO: 8 - protein, 5 aa, Drosophila melanogaster
IASGA

SEQ ID NO: 9 - protein, 6 aa, Drosophila melanogaster
ASIAGA

(19) United States
(12) Patent Application Publication
(10) Pub. No.: US 2009/0062150 A1
(43) Pub. Date: Mar. 5, 2009

(54) COMPOSITIONS AND METHODS FOR REGULATING INTRAMEMBRANE PROTEASES

(76) Inventor: Yigong Shi, Plainsboro, NJ (US)

Correspondence Address: PEPPER HAMILTON LLP, ONE MELLON CENTER, 50TH FLOOR, 500 GRANT STREET, PITTSBURGH, PA, 15219, US

(21) Appl. No.: 11/937,894

(22) Filed: Nov. 9, 2007

Related U.S. Application Data
(60) Provisional application No. 60/858,848, filed on Nov. 14, 2006.
(60) Provisional application No. 60/911,584, filed on Apr. 13, 2007.

(51) Int. Cl. A61K 38/08; C40B 60/04; C40B 30/02; C07K 7/06

(52) U.S. Cl. 506/35; 514/16; 506/8; 530/329

(57) ABSTRACT

Structural models for a rhomboid protease alone and bound to inhibitors and peptide substrates and compositions and methods for preparing rhomboid protease binding compounds and methods for using such rhomboid protease binding compounds for modulation of these proteases catalytic activity are disclosed herein.

### CROSS-REFERENCE TO RELATED APPLICATIONS

This application claims priority to and benefit of U.S. Provisional Application Ser. No. 60/857,848, entitled “A General Method of Regulating the Activity of Intramembrane Proteases”, filed Nov. 9, 2006; and U.S. Provisional Application Ser. No. 60/911,584, entitled “Compositions and Methods for Regulating Intramembrane Proteases”, filed Apr. 13, 2007 the entire contents of which are hereby incorporated by reference in its entirety.

### GOVERNMENT INTERESTS

Not Applicable

### PARTIES TO A JOINT RESEARCH AGREEMENT

Not Applicable

### INCORPORATION BY REFERENCE OF MATERIAL SUBMITTED ON A COMPACT DISC

Not Applicable

### BACKGROUND

1. Field of Invention

Intramembrane proteolysis is a signaling mechanism conserved in species ranging from bacteria to humans and plays an important role in cellular physiology. For example, the first description of intramembrane proteolysis came from an ER membrane bound transcription factor SREBP which is cleaved by an integral membrane protease, known as site-2 protease (S2P). As a result of this cleavage, the N-terminal domain of the SREBP which contains a DNA-binding domain and a trans-activation domain and regulates, transcription of a number of genes that control biosynthesis of cholesterol and fatty acids is released. Another example of intramembrane proteolysis is the proteolytic processing of the amyloid precursor protein (APP) by the intramembrane protease γ-secretase. The cleavage product of APP, amyloid β-peptide, exhibits pronounced toxicity to neuronal cells and is thought to contribute to Alzheimer's disease. More recently, a rhomboid protease has been identified as an essential component in the signal-sending cells during epidermal growth factor receptor (EGFR) signaling in Drosophila by cleaving the ligand Spitz, which is inactive in its full-length form.

2. Description of Related Art

To date, four families of intramembrane proteases have been identified: serine protease rhomboid, metalloprotease S2P, aspartyl proteases presenilin (catalytic subunit of γ-secretase), and signal-peptide peptidase. Rhomboids are a conserved family of intramembrane serine proteases which are involved in controlling diverse biological functions such as intercellular signaling, parasite invasion, quorum sensing, mitochondria morphology and dynamics, and apoptosis. Substrates for rhomboid proteases vary and include transmembrane proteins, such as, for example, EGF, TNFα, TGFα and other EGF receptor ligands, as well as thrombomodulin. The putative catalytic residues responsible for the protease activity of rhomboids are predicted to be below the membrane surface and within the hydrophobic core of the proteases.

### BRIEF SUMMARY OF THE INVENTION

Various embodiments of the invention described herein include a method for preparing a rhomboid protease modulating compound including the steps of applying a three-dimensional molecular modeling algorithm to the atomic coordinates of at least a portion of rhomboid protease; determining spatial coordinates of at least a portion of rhomboid protease; electronically screening stored spatial coordinates of candidate compounds against the spatial coordinates of at least a portion of rhomboid protease; identifying a compound that is substantially similar to at least a portion of rhomboid protease; and synthesizing the identified compound.

In some embodiments, the method may also include the step of identifying a candidate compound that deviates from the atomic coordinates of at least a portion of rhomboid protease by a root mean square deviation of less than about 5 angstroms. In other embodiments, the method may further include the step of testing the identified compound for binding at least a portion of rhomboid protease, and in certain embodiments, the method may further include the step of testing the identified compound for inhibiting rhomboid protease activity.

In various embodiments, the step of electronically screening stored spatial coordinates may further include identifying a compound that has a shape, a charge distribution, a size or a combination thereof substantially similar to a portion of rhomboid protease, and in some embodiments, the at least a portion of the rhomboid protease may be at least a, portion of one or more of: transmembrane helix 5 (TM5); transmembrane helix 4 (TM4); transmembrane helix 6 (TM6); loop 5 (L5); or loop 1 (L1). In such embodiments, the identified compound may inhibit entry of a substrate protein into an active site of the rhomboid protease, or in other such embodiments, the identified compound may enhance entry of a substrate protein into an active site of the rhomboid protease.

Some embodiments of the invention include a method for preparing a rhomboid protease inhibitor including the steps of: applying a three-dimensional molecular modeling algorithm to atomic coordinates of a rhomboid protease having a bound substrate peptide or applying a three-dimensional molecular modeling algorithm to atomic coordinates of a rhomboid protease having a rhomboid binding compound; determining spatial coordinates of at least a portion of substrate peptide or binding compound; electronically screening stored spatial coordinates of candidate compounds against the spatial coordinates of at least a portion of substrate peptide or binding compound; identifying a compound that is substantially complementary to the substrate peptide or binding compound; and synthesizing the identified compound.

In some embodiments, the method may further include the step of identifying a compound that has a, shape, a charge distribution, a size or a combination thereof substantially similar to at least a portion of the substrate peptide or binding compound, and in certain embodiments, the substrate peptide may be Spitz, C100-Spitz-Flag, a peptide derived form Toxoplasma gondii micronemal proteins, MIC2, or a combination thereof. In such embodiments, the identified compound may inhibit entry of substrate into an active site of the rhomboid protease.

In other embodiments, the method may further include the steps of: identifying one or more substrate peptides; isolating at least a portion of the one or more substrate peptides where the rhomboid protease is likely to bind the one or more substrate peptides; determining spatial coordinates of at least a portion of the one or more substrate peptides; and identifying a compound that is substantially similar to at least a portion of the one or more substrate peptides. In certain embodiments, the step of isolating one or more substrate peptides further include: identifying more than one substrate peptides; performing an alignment of the more than one substrate peptides; and isolating at least a portion of the more than one substrate peptides that share sequence similarity or secondary structure similarity.

Any of the methods described above may further include the step of testing the identified compound for binding to the rhomboid protease.

In particular embodiments, the synthesized compound may be one or more modified peptide bond, and in some embodiments, the synthesized compound may be modified such that rhomboid protease mediated proteolysis of the synthesized compound cannot occur. TN certain embodiments, the rhomboid binding compound may include one or more of: tetraethyleneglycol monoctyl ether (C8E4), dichloroisocourmarin (DCI) or a combination thereof.

Other embodiments of the invention include a pharmaceutical composition including an effective amount of a compound having a three-dimensional structure corresponding to atomic coordinates of at least a portion of a rhomboid protease, a substrate peptide of a rhomboid protease or a rhomboid protease binding compound and a pharmaceutically acceptable excipient or carrier, and in some embodiments, the compound may bind to the rhomboid protease.

Various other embodiments of the invention include a system for identifying rhomboid protease modulators including: a processor and a processor readable storage medium in communication with the processor readable storage medium comprising the atomic coordinates of at least a portion of rhomboid protease. In certain embodiments, the processor readable storage medium further include one or more programming instructions for: applying a three-dimensional modeling algorithm to the atomic coordinates of the rhomboid protease; determining spatial coordinates of at least a portion of the rhomboid protease; electronically screening spatial coordinates of candidate compounds with the spatial coordinates of at least a portion of the rhomboid protease; and identifying a candidate compound whose spatial coordinates are substantially similar to the spatial coordinates of at least a portion of the rhomboid protease or identifying a candidate compound whose spatial coordinates are substantially complementary to the spatial coordinates of at least a portion of the rhomboid protease.

In some embodiments, the one or more programming instructions for identifying a candidate compound whose spatial coordinates are substantially similar to the spatial coordinates of at least a portion of the rhomboid protease may include one or more programming instructions for identifying a compound that deviates from the spatial coordinates of at least a portion of the rhomboid protease by a user defined threshold. In other embodiments, the one or more programming instructions for identifying a compound whose spatial coordinates are substantially similar to at least a portion of the rhomboid protease may include one or more programming instructions for identifying, a compound having one or more of: a size within a user defined threshold; a charge within a user defined threshold; or a shape with a user defined threshold. In still other embodiments, the one or more programming instructions for electronically screening spatial coordinates of a candidate compound may include one or more programming instructions for simulating binding of the candidate compound to the rhomboid protease.

In further embodiments, the system may further include an output device in communication with the processor, and in some such embodiments, the processor readable storage medium may further include one or more programming instructions for: applying a three-dimensional modeling algorithm to the atomic coordinates of rhomboid protease; determining spatial coordinates of at least a portion of the rhomboid protease; generating a visual signal and relaying the visual signal to the output device; and electronically designing a compound that is substantially similar to at least a portion of the rhomboid protease or electronically designing a compound that is substantially complementary to at least a portion of the rhomboid protease.

Other embodiments of the invention are directed to a rhomboid protease binding compound including a molecule having a three-dimensional structure corresponding to atomic coordinates derived from at least a portion of an atomic model of a rhomboid protease, a rhomboid protease bound to an inhibitor or a rhomboid protease bound to a substrate.

In some embodiments, the inhibitor may be tetraethyleneglycol monoctyl ether (C8EA), dichloroisocourmarin (DCI) or a combination thereof, and other embodiments, the substrate may be Spitz C100-Spitz-Flag, a peptide derived form Toxoplasma gondii micronemal proteins, or a combination thereof.

In certain embodiments, the molecule may have a three-dimensional structure corresponding to atomic coordinates of at least a portion of tetraethyleneglycol monoctyl ether (C8E4), dichloroisocourmarin (DCI), Spitz, C100-Spitz-Flag, a peptide derived form Toxoplasma gondii micronemal proteins, or a combination thereof, and in certain other embodiments, the molecule may have a shape, a charge, a size or combinations thereof substantially: corresponding to a portion of a rhomboid protease.

In some embodiments, the molecule may bind at an interface between transmembrane helix 5 (TM5) or its structural equivalent in another rhomboid protease and another structural, feature of the rhomboid protease, and in others, the molecule may have a shape, a charge, a size or combinations thereof substantially complementary to a portion of a rhomboid protease. In some such embodiments, the molecule may inhibit access of substrate to the active site of the rhomboid protease. In yet other embodiments, the molecule may bind to at least a portion of the rhomboid protease with a greater affinity than a naturally occurring substrate, and in particular embodiments, the molecule may inhibit rhomboid protease mediated proteolysis. In yet further embodiments, the molecule may further include a pharmaceutically acceptable excipient or carrier.

In various embodiments, the molecule may deviate from the atomic coordinates of at least a portion of the rhomboid protease by a root mean square deviation of less than about 10 angstroms, and in particular embodiments, the molecule may deviate from the atomic coordinates of at least a portion of the rhomboid protease by a root mean square deviation of less than about 2 angstroms.

In at least one embodiment, the compound may be a peptide or peptidomimetic of sequence selected from:

(SEQ ID No. 1)

Ala-Gly-Ala-Ile-Ala-Gly-Gly,

(SEQ ID No. 2)

Ala-Ile-Ala-Gly-Gly-Val-Ile,

(SEQ ID No. 3)

Ala-Ile-Ala-Gly-Gly-Val-Val,

(SEQ ID No. 4)

Tyr-Tyr-Ala-Gly-Ala-Gly-Val,

(SEQ ID No. 5)

Ala-Gly-Ala-Ile-Ala-Gly-Gly,

(SEQ ID No. 6)

Ala-Gly-Ala-Ile-Ala-Gly-Gly-Val-Ile-Gly-Gly,

(SEQ. ID No. 7)

Ala-Ser-Gly-Ala,

(SEQ. ID No. 8)

Ile-Ala-Ser-Gly-Ala,
and

(SEQ. ID No. 9)

Ala-Ser-Ile-Ala-Gly-Ala.

### DESCRIPTION OF THE DRAWINGS

The file of this patent contains at least one drawing/photograph executed in color. Copies of this patent with color drawing(s)/photograph(s) will, be provided by the Office upon request and payment of the necessary fee. All figures where structural representations are shown were prepared using MOLSCRIPT (Kraulis (19913) J Appl Crystallogr 24:946-950) and GRASP (Nicholls et al. (1991) Proteins: Struct Funct Genet 11:281-296).

For a fuller understanding of the nature and advantages of the present invention, reference should be made to the following detailed description taken in connection with the accompanying drawings, in which:

FIG. 1A illustrates results of an in vitro assay for the enzymatic activity of the wild type GlpG core and an active site mutant, S201A.

FIG. 1B illustrates results of an in vitro assay for the enzymatic activity of the full length GlpG protease and the GlpG core against the substrates C100Spitz-Flag and C100-Flag.

FIG. 1C is a schematic diagram illustrating the overall structure of GlpG in one asymmetric unit.

FIG. 1D is a schematic diagram illustrating the structure of Molecule A in the asymmetric unit.

FIG. 2 is an alignment of rhomboid homologs from several species. Secondary structural elements of GlpG are indicated above the sequences and conserved amino acids are highlighted.

FIG. 3A is a schematic diagram of Molecule A showing the open cavity leading to the active site.

FIG. 3B is a stereo diagram of interactions surrounding the Trp-Arg motif of the rhomboid proteases (W136-R137).

FIG. 3C is a stereo diagram of interactions between residues of loop L1 and transmembrane helix α31 (TM3) and loop L3.

FIG. 3D is a stereo diagram overlay of the GlpG structure described herein and a previous model depicting interactions surrounding the L1 loop.

FIG. 4A illustrates results of an in vitro assay for the enzymatic activity of the GlpG core with amounts of the detergent inhibitor C8E4.

FIG. 4B is a schematic diagram of the overall structure of GlpG bound to C8E4 in one asymmetric unit.

FIG. 4C is a stereo diagram of conformations of TM5′ in Molecule A (dark) and B (light).

FIG. 4D is a stereo diagram of C8E4 (cage) bound to the GlpG core (wire).

FIG. 5A is an overlay of GlpG bound to C8E4 (light) or DCI (dark), or soaked in MIC2 substrate (very light).

FIG. 5B is a stereo diagram of electron density surrounding residue Ser201 of Molecule B in the presence of DCI.

FIG. 5C is a stereo diagram view of electron density surrounding DCI in Molecule B of DCI soaked crystals.

FIG. 5D is a stereo diagram of the active site in the structure of Molecule B of MIC2Z soaked crystals.

FIG. 6A is a stereo diagram of an overlay of the atomic models for GlpG described herein and another four published GlpG atomic models.

FIG. 6B is a stereo diagram overlay of the L1 loop connecting transmembrane helices α1 and α2 (TM1 and TM2) of the atomic models for GlpG described herein and another four published GlpG atomic models.

FIG. 6C is a stereo diagram of an overlay of the TM5 region of the atomic models for GlpG described herein and another four published GlpG atomic models.

FIG. 7 is a surface representation of the atomic models for GlpG described herein and another four published GlpG atomic models.

FIG. 8A is a diagram illustrating the position of residues targeted for mutagenesis within the GlpG atomic model.

FIG. 8B illustrates results of an in vitro assay for the enzymatic activity of mutants of the GlpG core: wild-type GlpG (WT); L143C; F127C P195C; H141C G198C.

FIG. 8C illustrates results of an in vitro assay for the enzymatic activity of mutants of the GlpG core which destabilize the interaction between TM5 and TM2: wild-type GlpG (WT); W236C F153C; W236A F153A.

FIG. 8D is a stereo diagram of a putative substrate peptide bound to the GlpG.

### DETAILED DESCRIPTION

Before the present compositions and methods are described, it is to be understood that they are not limited to the particular compositions, methodologies or protocols described, as these may vary. It is also to be understood that the terminology used in the description is for the purpose of describing the particular versions or embodiments only, and is not intended to limit their scope in the present disclosure which will be limited only by the appended claims. Various scientific articles, patents and other publications are referred to throughout the specification. Each of these publications is incorporated by reference herein in its entirety.

It must also be noted that as used herein and in the appended claims, the singular forms “a”, “an”, and “the” include the plural reference unless the context clearly dictates otherwise. Thus, for example, reference to an “inhibitor” is a reference to one or more inhibitors and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments disclosed, the preferred methods, devices, and materials are now described.

“Optional” or “optionally” means that the subsequently described event or circumstance may or may not occur, and that the description includes instances where the event occurs and instances where it does not.

Throughout the specification of the application, various terms are used such as “primary”, “secondary”, “first”, “second”, and the like. These terms are words of convenience in order to distinguish between different elements, and such terms are not intended to be limiting as to how the different elements may be utilized.

As used herein, “isolated” means altered or removed from the natural state through human intervention. For example, a rhomboid protease naturally present in a living animal is not “isolated,” but a synthetic rhomboid protease, or a rhomboid protease partially or completely separated from the coexisting materials of its natural state is “isolated.” An isolated rhomboid protease can exist in substantially purified form, or can exist in a non-native environment such as, for example, a cell into which the rhomboid protease has been delivered.

The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics, as further described below.

By “pharmaceutically acceptable”, it is meant the carrier, diluent or excipient must be compatible with the other ingredients of the formulation and not deleterious to the recipient thereof. As used herein, the term “pharmaceutically acceptable salts, esters, amides, and prodrugs” refers to those carboxylate salts, amino acid addition salts, esters, amides, and prodrugs of the compounds of the present disclosure which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention.

The terms “therapeutically effective” or “effective”, as used herein, may be used interchangeably and refer to an amount of a therapeutic composition of embodiments of the present invention (e.g. one or more of the peptides or mimetics thereof). For example, a therapeutically effective amount of a composition comprising a mimetic is a predetermined amount calculated to achieve the desired effect. As used herein, an “effective amount” of an antagonist or mimetic is an amount sufficient to cause antagonist mediated inhibition of rhomboid protease, and thus modulate rhomboid protease activity in a range of disorders, such as those related to insulin resistance and blood coagulation, inflammatory disorders, neurodegenerative and cardiovascular diseases, apoptosis, cancer and early-onset blindness.

The invention presented herein is generally directed to the atomic coordinates of GlpG, methods for using the atomic coordinates of GlpG, small molecules and mimetics prepared using such methods, and methods for using such small molecules and mimetics to modulate the activity of the Rhomboid family of intra-membrane proteases. In particular, high resolution crystal structures of GlpG, an E. coli rhomboid protease, high resolution crystal structures of GlpG in the presence of a substrate peptide, and high resolution crystal structures of GlpG in the presence of an inhibitor are provided herein. Various embodiments of the invention include methods for using the atomic coordinates of any of the GlpG crystal structures described herein or any rhomboid protease for screening a library of compounds of known structure for the ability to bind to and modulate the activity of rhomboid proteases and rational design modulators rhomboid proteases. Other embodiments include peptides, small molecules, mimetics, and the like that modulate the activity of rhomboid proteases designed or identified using such methods. Still other embodiments include therapeutic agents prepared from such modulators.

As used herein, the term “modulator” may be used to define a compound that modifies the activity of a rhomboid protease. For example, in some embodiments, a modulator may inhibit function of a rhomboid protease. Such modulators may also by considered antagonists. In other embodiments, a modulator may activate or stimulate activity of a rhomboid protease. Such modulators may also be considered agonists.

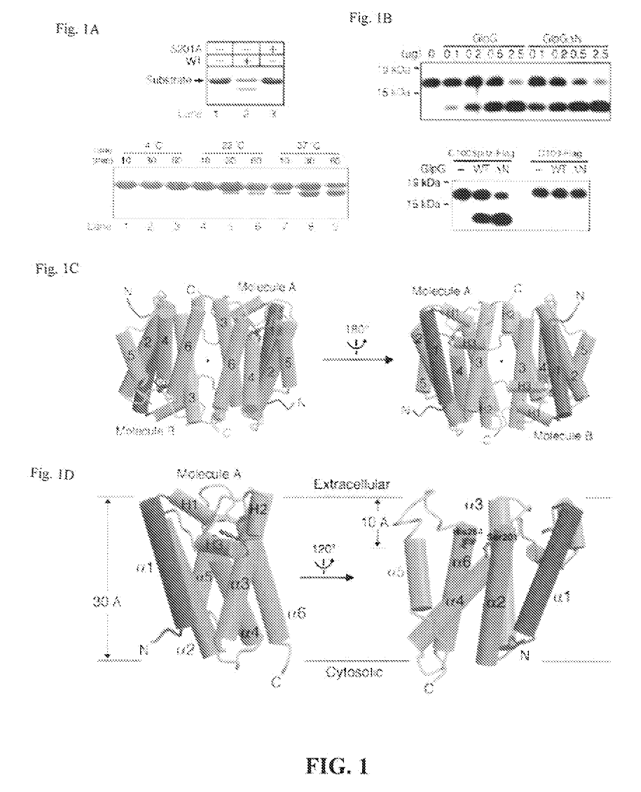

Structural characterization of E. coli rhomboid protease, GlpG, was carried out using an N-terminally truncated GlpG transmembrane core domain (residues 87-276). In vitro characterization of the enzymatic activity of the truncated GlpG was reconstituted to validate the use of the GlpG core domain for crystallographic studies was carried out using two separate assays. Proteolytic activity of purified truncated GlpG core domain on an artificial protein substrate in detergent micelles was analyzed at 37° C. As illustrated in FIG. 1A, top panel, the truncated GlpG actively catalyzed proteolysis of the artificial substrate. However, a mutation at Serine 201 (S201A) abolished the observed proteolytic activity of purified truncated GlpG core domain. FIG. 1A, bottom panel shows proteolysis of the artificial substrate over time at 4° C., 22° C. and 37° C. and indicates that the proteolytic activity of purified truncated GlpG occurs at 22° C. but not at 4° C. and may be improved at 37° C. Proteolytic activity of purified truncated GlpG core domain was also compared with full length GlpG using C100-Spitz-Flag as substrate. FIG. 1B, top panel shows that truncated GlpG core domain catalyzes proteolysis of the C100-Spitz-Flag at a similar level of activity to lull-length GlpG. Moreover, neither full-length GlpG nor truncated GlpG was able to cleave C100-Flag as illustrated in FIG. 1B, bottom panel. This suggests that substrate specificity for the Spitz transmembrane domain is maintained in the truncated GlpG transmembrane domain.

Crystals of truncated GlpG core domain (hereinafter “GlpG”) were prepared under physiological pH (pH 7.4) and normal ionic strength. These crystals diffracted X-rays to a resolution of 2.6 Å at synchrotron sources. A native data set was collected and heavy atom derivatives were prepared. Atomic coordinates for GlpG were determined using the collected data and deposited with the protein data bank (PDB) under accession code 2NRF (See Table 1 for crystallographic data). Each asymmetric unit of the crystal contains two molecules of GlpG (Molecules A and B), which form a pseudo-dimer as shown in FIG. 1C. The overall structure of these two molecules has a root-mean-square deviation (rmsd) of about 0.5 Å over 167 aligned Cα carbons out of a total of 190 atoms.

The arrangement of molecule A within the lipid bilayer is shown in FIG. 1D. As predicted, the structure of the GlpG contains six α-helices arranged essentially perpendicular to the surface of the lipid bilayer. Transmembrane helices α1, α2 and α3 (TM1, TM2 and TM3) contain 20, 22 and 23 residues, respectively, and are likely to traverse the entire lipid bilayer. TM1, TM2, and TM3 and the N-terminal portion of transmembrane helix α4 (TM4) stack against one another and form extensive van der Waals interactions which are further buttresses by an extended loop (L1) between helices TM1 and TM2. The L1 loop is made up of three short α-helices (H1, H2 and H3) that stack against hydrophobic residues on TM3. These extensive networks of structural features associated with TM1, TM2, TM3, and L1 suggest that the N-terminal of GlpG may constitute a structural scaffold on which the rest of the molecule functions.

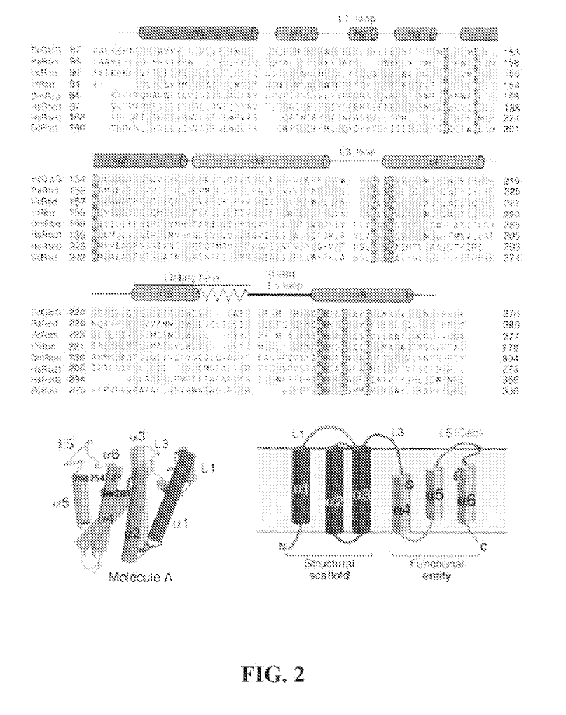

FIG. 1D, right panel shows the transmembrane helices α4, α5 and α6 (TM4, TM5 and TM6) which are shorter than TM1, TM2 and TM3 and, therefore, may not traverse the entire lipid bilayer. FIG. 2, lower right panel further illustrates the arrangement of helices within the lipid bilayer. It is of note, that putative catalytic residues, such as, for example, S201, may be located at the N-terminus of TM4 and appears to be positioned approximately 10 Å below the membrane surface. TM6 may contain other catalytic residues, such as, for example, histidine 254 (H254) which may donate a hydrogen to S201 during catalysis. TM5 may not contain a catalytic residue, but has considerable conformational flexibility. Taken together, the shorter lengths of the TM4, TM5 and TM6 and the presence of catalytic residues suggests that the C-terminal half of GlpG may constitute a functional component of the rhomboid protease that catalyzes the scission of peptide bonds.

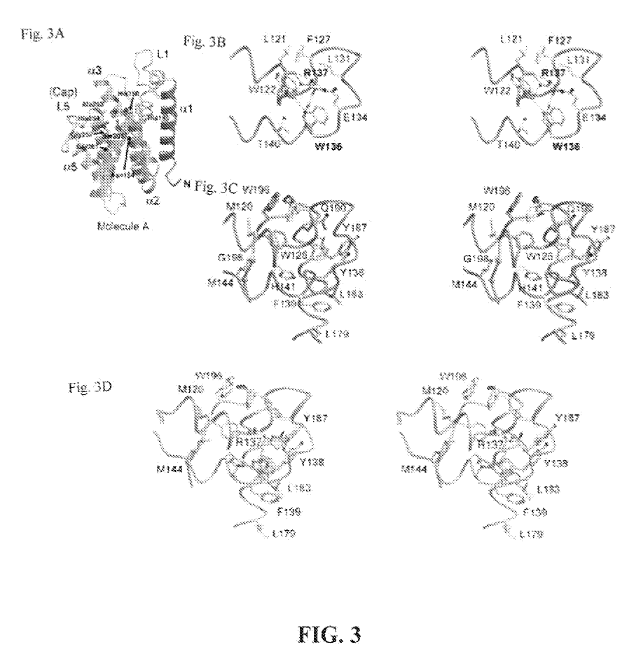

FIG. 3A shows a cavity located at the bottom of a V-shaped funnel which appears to open to the extracellular side of the lipid bilayer which may include the active site of GlpG. This, cavity appears to be formed by the N-terminal portion of TM2, the C-terminal portions of TM3 and TM5, a loop (L3) linking TM3 and TM5 and a loop (L5) linking TM5 and TM6. An alignment of eight (8) rhomboid protease homologs as shown in FIG. 2: indicates ten invariant transmembrane residues which are highlighted. These residues include four glycines (G199, G202, G257 and G261), three histidines (H145, H150 and H254), one serine (S201), one asparagine (N154), and one alanine (A253), and the position of each of these residues within the cavity or in close proximity to the cavity are illustrated in FIG. 3A using a dot. This may allude to a functional significance of these residues as well as the cavity in general.

This proposed active site location in the cavity described above is in agreement with the previously reported structures of E. coli GlpG and H. influenzae GlpG which propose that a catalytic serine residue, (S201) is located at the bottom of a funnel-shaped cavity that opens to the extracellular side of the protease. Additionally, three well ordered water molecules located within the cavity, one of which makes a hydrogen bond to the hydroxyl oxygen atom of a putative catalytic residue S201, may be important in stabilizing these catalytic residues. Additionally, the position of H254, another proposed catalytic residue, in the structure shown in FIG. 3A which may stabilize S201 through hydrogen bonding to H254 is in good agreement with the previously proposed structures.

This cavity of GlpG appears to be considerably larger than a similar cavity of previous structural models of GlpG (Wang et al. (2006) Nature 444:179-180). This appears to largely be due to the position of TM5 which is away from the rest of the molecule. These previous models suggest that L5 forms a “cap” over the cavity closing the cavity to solvent. In contrast, the position of TM5 in the model described herein is away from the opening and may allow the cavity, and hence the active site, to be open to solvent. Additionally, the size of the cavity of Molecule B appears to be smaller than the cavity of Molecule A; and L5 of Molecule B appears to have little to no electron density. This may indicate that L5 has a high degree of flexibility. This observation may provide additional evidence that the position of L5 may vary depending on the activity of the protease. For example, L5 may shift to apposition indicated by Molecule A allowing the cavity to be “open” when the protease is active, and L5 may be positioned to form a “cap” over the cavity when the protease is inactive.

Loop L3 may also be an important element of the cavity. L3 appears to stack against extended loop L1 and TM3. As illustrated in FIG. 3C, hydrophobic residues in L1 appear to stack against non-polar residues in L3 and the C-terminal half of the TM3 through a myriad of van der Waals interactions. The extensive packing interactions amongst residues of L1, also include two highly conserved residues, tryptophan 136. (W136) and arginine 137 (R137), which participate in a network of hydrogen bonds as illustrated in FIG. 3B. At the center of the L1 loop, the guanidium group of R137 appears to donate five hydrogen bonds to neighboring residues: two charge stabilized contacts to glutamate 134 (L134) and three hydrogen bonds to backbone carbonyl oxygen atoms of residues luecine 121 (L121) and arginine 122 (R122). The carbonyl oxygen atom of R122 accepts an additional hydrogen bond from the side chain of W136. In addition, R137 makes a number of van der Waals, contacts with surrounding residues in loop L1. These observations predict that mutation of R137 and to a lesser extent mutation of W136 may compromise the structural stability of rhomboid proteases, and may, therefore, cause a reduction in proteolytic function. The extensive interactions both within L1 and between L1 and other structural elements of GlpG identified above are essentially identical to previously reported as illustrated by the overlay presented in FIG. 3D. In fact, the main chain as well as a vast majority of the side chains in L1 and TM3 have identical conformations in both structures.

Despite structural similarities for the atomic models, contrasting models have been proposed to explain substrate entry into the active site of rhomboid proteases. In a first model, extended L1 between TM1 and TM2 forms a lateral gate responsible for substrate entry. In this model, L1 opens during catalysis to allow substrate to enter the active site between TM1 and TM3. In a second model, TM5 serves as a gate for substrate entry. Therefore, the structure of GlpG in, the previous model is in “closed” conformation because TM5 is acting as a “cap” for the active site cavity. The structure of the rhomboid protease described herein is in “open” conformation because TM5 is shifted away from the active site cavity allowing substrate entry between TM5 and TM2. Structures of GlpG bound to inhibitors and substrate peptides were also determined, and analysis of these structures in comparison to the unbound GlpG provide strong evidence that substrate entry into the active site cavity of rhomboid proteases occurs between TM5 and TM2 and the active site is gated by TM5 as suggested by the second model.

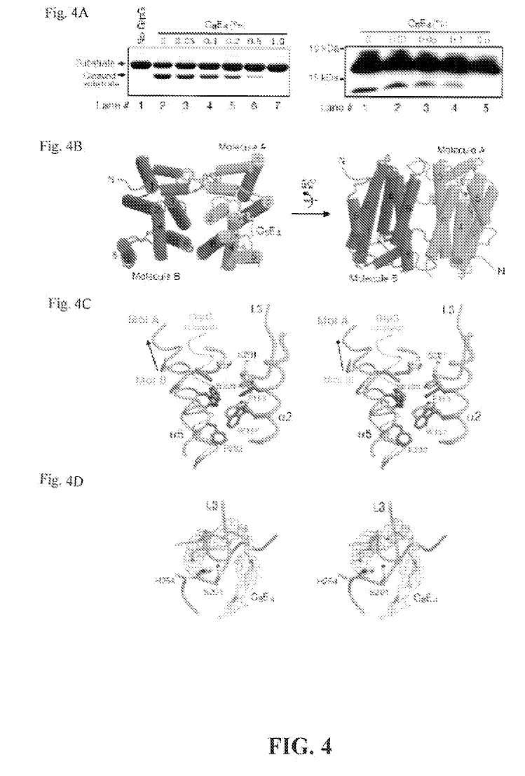

Detergent molecules are known to inhibit enzymatic activity of rhomboid proteases. Detergent tetraethyleneglycol monooctyl ether (C8E4), at concentrations above its critical micelle concentration (CMC) appears to inhibit enzymatic activity of the truncated GlpG transmembrane core domain on artificial substrate as illustrated in FIG. 4A, left panel, and this inhibition is also observed for proteolysis C100-Spitz-Flag as shown in FIG. 4A, right panel. This inhibitory activity appears to be specific to C8E4 as a number of other detergents, including nonyl glucoside and LDAO, exhibited no inhibition of GlpG over a wide range of concentrations up to several times their CMCs (data not shown).

Inhibition of GlpG by C8E4 was characterized by crystallizing the GlpG in the presence of C8E4 under conditions nearly identical to that reported hereinabove. The crystallographic structure of the GlpG-C8E4 was determined by molecular replacement and refined at 3.0 Å resolution as shown in FIG. 4B, and the inhibition of GlpG byC8E4 was characterized. Each asymmetric, unit of GlpG-C8E4 appears to contain two molecules of GlpG, designated Molecule A and Molecule B which exhibit nearly identical conformations throughout the structure except the TM5 region, which differs significantly. As illustrated in FIG. 4C, TM5 in Molecule A is considerably closer to TM6 than in Molecule B. However, TM5 in both Molecule A and Molecule B of GlpG-C8E4 appear to be 5-10 Å further away from TM2 than in unbound GlpG in “closed” conformation, and the gap between TM5 and TM2 may be sufficient for binding to substrate. Therefore, despite the differences between Molecule A and Molecule B of GlpG-C8E4, both molecules may be in “open” conformation.

FIG. 4D shows the elongated C8E4 molecule forming an arch directly above S201 in Molecule. A. One side of the C8E4 arch appears to block H254 and the other side may block the backbone of L3. Additionally, the hydrocarbon end of C8E4 is; within van der Waals contact distances of V204, Y205, F232, and W236. Based on this arrangement, access of substrate to the catalytic S201 may be blocked providing a plausible explanation to the C8E4-mediated inhibition of GlpG.

GlpG-C8E4 crystals appear to exhibit improved stability over crystals of GlpG alone. Moreover, Molecule B in the asymmetric unit appears to be in an “open” conformation but does not appear to contain bound C8E4. Therefore, GlpG-C8E4 crystals were soaked in solutions containing inhibitors or substrate peptides of GlpG with the expectation that an inhibitor or substrate peptide may bind to the open conformation active site of Molecule B in the stabilized GlpG-C8E4 crystals. Dichloroisocoumarin (DCI) is a relatively potent inhibition of GlpG and DCI bound GlpG-C8E4 crystals were obtained by soaking GlpG-C8E4 crystals with 5-10 mM DCI for about 10 minutes. Diffraction data was collected and a structural model for the DCI bound GlpG-C8E4 crystals was determined at 3.0 Å resolution. Additional crystallographic statistics are provided in Table 1.

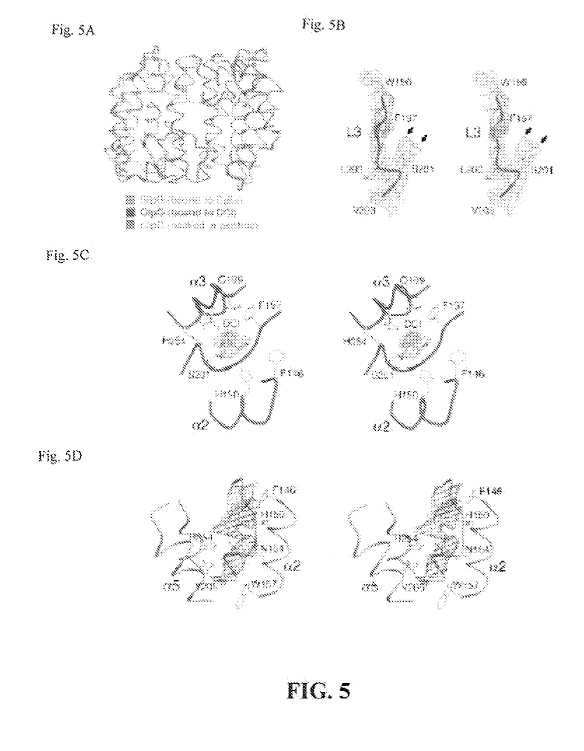

Based on the atomic model of DCI bound GlpG-C8E4, DCI appear to covalently bind to the Oγ atom of S201 in Molecule B. As can be observed in FIG. 5B, the shape and size of the electron density surrounding S201 are consistent with the presence of a DCI molecule bound to S201. Additionally, the model of DCI covalently linked to S201, as provided in FIG. 5C, provides a similar electron density indicating that the added electron density surrounding S201 in the atomic model of DCI bound GlpG-C8E4 is DCI covalently bound to S201. Additionally FIG. 5C also shows that the hydrophobic ring of DCI appears to make van der Waals contacts with histidine 150 (H150), phenolalanine 197 (F197), alanine 253 (A253), histidine 254 (H254), and the aliphatic portion of glutamic acid 189 (Q189). Without wishing to be bound by theory, because DCI can be soaked into GlpG-C8E4 crystals, inhibitors may be able to gain access to the active site of GlpG when it is in its open conformation. This observation further suggests that a substrate peptide may be able to approach the active site or GlpG between TM5 and TM2.

GlpG-C8E4 crystals were also soaked with known substrate peptides of rhomboid proteases. Crystals of diffraction quality were obtained from GlpG-C8E4 crystals soaked in about 2-5 mM of a 7-mer (AGAIAGG, SEQ. ID. No. 1) derived from Toxoplasma gondii micronemal proteins (MIC2). Diffraction data for these crystals was collected and an atomic structure for MIC2 bound GlpG-C8E4 was refined to 2.6 Å resolution. Additional crystallographic statistics are provided in Table 1.

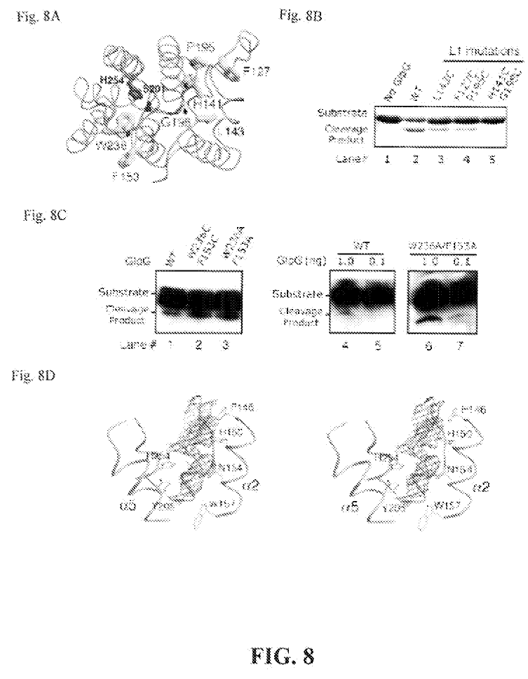

FIG. 5D shows an elongated stretch of electron density close to S201 and between TM5 and TM2 in Molecule B in the atomic model of MIC2 bound GlpG-C8E4. This electron density appears to be absent in both the atomic models of GlpG-C8E4 alone and DCI bound GlpG-C8E4 suggesting that the additional electron density may represent the MIC2 peptide. It is also noted that the quality of the electron density may be consistent with the transient nature of the interaction between the active site of GlpG and the putative substrate peptide. FIG. 8D shows a more detailed atomic model of the MIC2 peptide, shown as a wire diagram with electron density cage, bound in the GlpG active site, shown as a wire diagram. The positioning of the substrate in the active site puts the scissile peptide bond (AGAIA-GG) above the putative catalytic residue (S201). The hydrophobic isoleucine side chain appears to contact with F146 and H150, and the other amino acids, N154, W157, Y205 and H254, shown in FIG. 8D may also be in position to contact the substrate peptide and act to position and/or hold the substrate in place.

FIG. 5A shows an overlay of the atomic models of DCI bound GlpG-C8E4, MC2 bound GlpG-C8E4 and GlpG-C8E4 alone. The overall structures of these atomic models are nearly identical. In fact, the rmsd of DCI bound GlpG-C8E4 in comparison to GlpG-C8E4 alone is about 0.59 Å over 355 backbone Cα atoms, and the rmsd of MC2 bound GlpG-C8E4 in comparison to GlpG-C8E4 alone is 0.6 Å over 354 backbone Cα atoms.

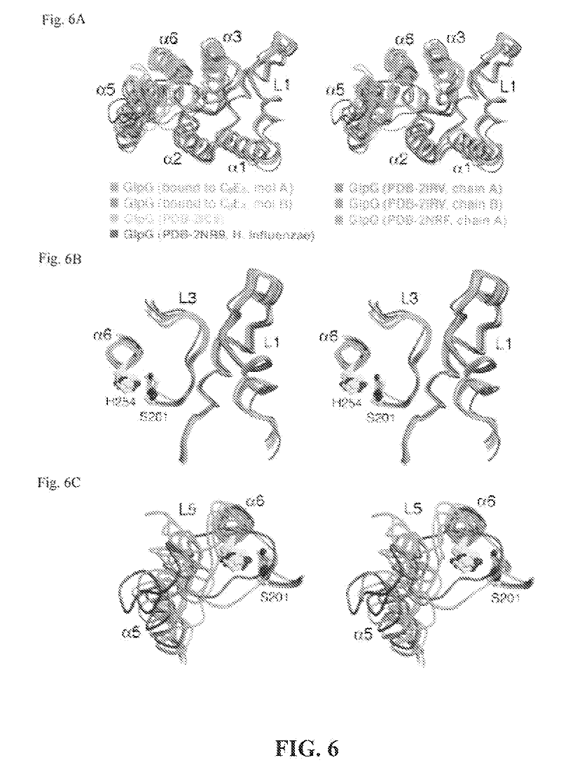

FIG. 6A shows an overlay of seven rhomboid protease structures including: one molecule crystallized in the R32 space group (PDB code 2IC8), two molecules of GlpG in the P21 space group (PDB code 2IRV), and GlpG homolog from Haemophilus influenzae in P212121 space group (PDB code 2NR9), as well as Molecules A and B of GlpG-C8E4, and Molecule A of GlpG alone (2NRF). Compared with Molecule A of GlpG-C8E4, the structure from PDB-2IC8 superimposed with an rmsd of 0.74 Å over 160 backbone, Cα atoms (residues 91-227 and 250272); the two molecules A and B of GlpG from PDB-2IRV were superimposed with rmsd's of 0.88 Å and 0.82 Å, respectively, over 171 C□ atoms (residues 92-239 and 250-272). A GlpG homolog from Haemophilus influenzae was superimposed with an rmsd of 1.24 Å over 164 backbone Cα atoms (residues 91-222, 229-238, and 250271). This analysis suggests that the structural elements of GlpG are, similar for each of the seven. GlpG atomic models compared in pair-wise comparisons which may indicate structural similarity amongst diverse members of the Rhomboid family of proteases. For example, as indicated by the overlay of FIG. 6B, the structural coordinates of L1 are similar among each of the seven GlpG species compared, and this degree of structural similarity is comparable to other regions of the structure. In fact, even the GlpG homolog from Haemophilus influenzae, the most distant member of the seven species, shows significant similarity.

In contrast, as illustrated by the overlay of FIG. 6C, the TM5 region of each of the seven GlpG species compared appears to deviate significantly. For any pair-wise comparison among the seven GlpG species aligned, the degree of structural variation is much greater in the TM5 region than in any other structural element including L1. For example, alignments of various GlpG species can exhibit a backbone shift by as much as about 6 Å in the TM5 region and only up to about 2 Å in L1.

This degree of variation in the TM5 region suggests that TM5 may exhibit several distinct conformations which may have been induced by various crystallization conditions. In contrast, L1 may adopt a more rigid structure of L1 since its structural conformation tends to adopt a more similar structure. Without wishing to be bound by theory, TM5 may adopt different conformations because it is inherently flexible. Moreover, this flexibility may provide a means for a gating mechanism via TM5, and this coupled with the apparent rigidity of the L1 structure provides evidence that L1 may not provide the gatin mechanism as previously reported. The various conformations of TM5 observed in the atomic structures described above may represent stages of gate opening that may be required for rhomboid function.

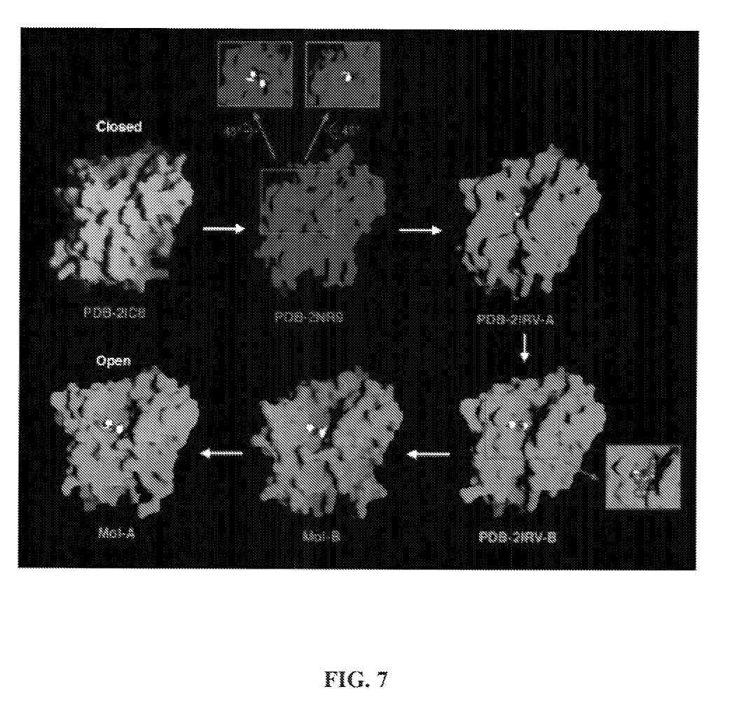

FIG. 7 shows a surface representation of the seven rhomboid species. A comparison of the location of TM5 and the apparent availability of the active site groove suggests that the rhomboid species represented by PDB-2IC8 may display a completely closed conformation wherein access to the active site is completely or almost completely blocked. Four rhomboid species, two represented by PDB-2IRV-A and PDB-2IRV-B and molecules A and B of GlpG-C8E4, may exhibit a more open conformation providing at least some access to the active site although the degree of opening appears to vary slightly. The rhomboid species represented by PDB-2NR9 also appears to be in a partially open conformation. However, the opening appears to be in the direction of transmembrane helices (see, insets).

Limited mutagenesis confirms the results described above. Targeted mutagenesis was carried out at amino acids though to destabilize L1. In all, three GlpG mutants, L143C, F127C/P195C, and H141C/G198C were generated in which four residues, H141 and G198 which appear to be buried and F127 and P195 which are solvent-exposed, are located at the interface between the L1 and the L3 loops. L143 appears to contribute to van der Waals interactions in the L1 loop. The location of each of the mutated amino acids in the atomic model of GlpG is shown in FIG. 8A. The mutant proteins were purified to homogeneity and their protease activity was examined. As indicated in FIG. 8B, compared to the wild-type GlpG protein, the three GlpG mutants exhibited compromised enzymatic activity. In fact, H141C/G198C exhibits virtually no proteolytic activity (lane 5). These observations are not consistent with the hypothesis that the L1 loop is a lateral gate: for substrate entry because mutations that either destabilize, L1 or weaken the interaction between L1 and neighboring structural elements of GlpG should increase the mobility of L1 and hence increase the enzymatic activity of GlpG.

Mutations in the TM5 region were also examined. Lateral access to the catalytic residues S201 and H254 is blocked by two aromatic residues, W236 on TM5 and F153 on TM2. In the closed form of GlpG, W236 and F153 interact with each other through van der Waals contacts. The position of these amino acids in the atomic model of GlpG is provided in FIG. 8A. Two GlpG mutants, W236C/F153C and W236A/F153A, were generated and purified to homogeneity. As shown in FIG. 8C, both mutants appear to exhibit significantly higher activity than the wild-type GlpG protein (lane 1-3). Moreover, a titration of GlpG appears to indicate that the mutant W236A/F153A is approximately 10 times more active than wild-type GlpG (lanes 4-7). These results may suggest that TM5 is the gate, thus mutation of W236 and F153 to smaller amino acids may destabilize the interaction between TM5 and TM2 increasing the enzymatic activity of GlpG. Additionally, W236A/F153A appear to exhibit higher activity than W236C/F153C which may suggest that mutating W236/F153 to the smaller alanine residues may facilitate substrate entry into the active site of GlpG. Taken together, the mutagenesis data may suggest that TM51 and not the L1 loop, serves as a gate regulating substrate entry into GlpG.

TABLE 1
Crystallographic Data Collection Statistics

| | GlpG | Bound to C8E4 | Bound to DCI | Soaked in peptide |
|---|---|---|---|---|
| Space group | P3 (1) | | | |
| Resolution (outer shell) | 50-2.6 (2.69-2.60) | 100-3.0 (3.11- | 100-3.0 (3.11- | 100-2.60 (2.69- |
| Unique observations | | 9,465 | 9,383 | 13,707 |
| Data redundancy (outer | 9.0 (6.3) | 4.8 (4.3) | 4.5 (3.4) | 2.3 (2.2) |
| I/sigma (outer shell) | 32.0 (2.1) | 13.5 (2.39) | 22.2 (1.45) | 16.7 (1.24) |
| Data coverage (outer | 98.5% (93.9%) | 99.6% (97.3%) | 99.6% (97.7%) | 95.1% (82.5%) |
| R_{sym} (outer shell) | 0.071 (0.508) | 0.112 (0.551) | 0.079 (0.615) | 0.070 (0.419) |

Refinement

| | | | | |
|---|---|---|---|---|
| Resolution (outer shell) | 30.0-2.6 | 20-3.0 (3.13- | 20-3.0 (3.13- | 20-2.6 (2.71-2.60) |
| Number of reflections | 13,455 | 8,956 | 8,864 | 12,941 |
| Data coverage | | 99.74% | 99.63% | 95.39% |
| R_{work} (outer shell) | | 0.262 (0.329) | 0.251 (0.296) | 0.247 (0.299) |
| R_{free} (outer shell) | | 0.306 (0.463) | 0.299 (0.385) | 0.289 (0.306) |
| R_{work}/R_{free} | 0.274/0.290 | | | |
| Total number of atoms | 2861 | 2935 | 2876 | 2914 |
| Protein | 2835 | 2935 | 2876 | 2914 |
| Ligand/ion | 0 | 0 | 0 | 0 |
| Water | 26 | 0 | 0 | 0 |
| B-factors | 96.61 | | | |
| Protein | 96.95 | | | |
| Water | 58.93 | | | |
| R.m.s.d. bond length | 0.012 | 0.006 | 0.007 | 0.012 |
| R.m.s.d. bond angles | 1.84 | 0.932 | 1.067 | 1.541 |

R_{sym} = ΣₕΣᵢ|I_{h,i} − Iₕ|/ΣₕΣᵢ I_{h,i}, where Iₕ is the mean intensity of the i observations of symmetry related reflections of h.
R = Σ|F_{obs} −F_{calc}|/ΣF_{obs}, where F_{obs} = F_{P}, and F_{calc} is the calculated protein structure factor from the atomic model (R_{free} was calculated with 5% of the reflections).
R.m.s.d. in bond lengths and angles are the deviations from ideal values.

The atomic model of the GlpG core domain provided herein suggest that entry of substrates into the active site of rhomboid proteases may occur through a conformational shift in transmembrane helix α5 (TM5) leading to the opening of a substrate pocket. Movement of the TM5, therefore, may act as a gate regulating the entry of substrate molecules into the active site and thus modulating protease activity of the rhomboid protease. The movement of TM5 may be characterized by a bending of TM5 in an outward direction, away from the core of the molecule. In this “open” state, a substrate pocket with a top lateral region that is large enough to accommodate a polypeptide chain of the substrate is created.

Various embodiments of the invention include modulators of rhomboid proteases prepared using the crystallographic data presented herein. Various other embodiments of the invention include methods for preparing such modulators and methods for identifying such modulators. Modulators of rhomboid proteases may “modulate” “change,” or modify the activity of a rhomboid protease in any way. For example, in some embodiments, the modulator of rhomboid protease may inhibit a rhomboid protease reducing the proteolytic activity of the protein, and in other embodiments, the modulator may activate the rhomboid protease increasing its proteolytic activity. In still other embodiments, the modulators may be useful for therapeutic applications, and in yet other embodiments, the modulators of the invention described herein may be prepared as pharmaceutical compositions.

Modulators of rhomboid protease activity, in some embodiments, may restrain movement of a one or more structural element of the rhomboid protease. For example, in one embodiment, the modulator may bind TM5 or a corresponding element of a rhomboid protease that acts as a gating element for substrate entry, and restrain the movement of this element. In other embodiments, modulators may increase the flexibility of a structural element of a rhomboid protease by, for example, interfering with inter or intramolecular bonds within the protein. Without wishing to be bound by theory, modulators that restrain or enhance flexibility of the gating element may increase or decrease proteolytic activity of the rhomboid protease.

The modulators encompassed by embodiments of the invention may be prepared or identified by applying a three-dimensional molecular modeling algorithm to the atomic coordinates of GlpG, GlpG-C8E4, DCI-bound GlpG-C8E4, peptide-bound GlpG-C8E4 or a composite of the atomic coordinates of these compounds or a, composite of these compounds' other rhomboid proteases. In certain embodiments, atomic coordinates defining a three-dimensional structure of the GlpG may be those with protein data bank accession code 2NRF. The molecular model prepared may then be used to identify molecules substantially complementary at least a portion of the surface of the rhomboid protease or to electronically screening stored spatial coordinates of candidate compounds against the atomic coordinates of the active site to identify candidate compounds that mimic at least a portion of the rhomboid protease or a peptide or inhibitor bound to the rhomboid protease. Compounds so identified may then be synthesized and tested for binding to the rhomboid protease. Compounds that are found to bind the rhomboid protease may then be used to determine their activity in inhibiting or activating the rhomboid protease.

In some embodiments, a portion of the atomic coordinates of a rhomboid protease or a portion of composite coordinates may be used to identify rhomboid protease binding compounds. For example, in one embodiment, the active site or a portion of the rhomboid protease thought to contain the active site may be isolated and used in the methods described above. In a particular embodiment, the active site may at least include the atomic coordinates of amino acids S201 and H254 or a corresponding residue in another rhomboid protease. In another embodiment, the atomic coordinates of all or a portion of a substrate peptide or an inhibitor bound to the rhomboid protease may be used to design or identify compounds that mimic their structure or provide additional molecular contacts that may enhance binding to the rhomboid protease. Compounds identified in methods using the coordinates of the active site or area surrounding the active site may bind to the active site and inhibit substrate entry in to the active site. Such compounds would be considered inhibitors.

In other embodiments, a portion of the atomic coordinates of a rhomboid protease or a portion of composite coordinates defining a gating element of a rhomboid protease may be identified and used to identify compounds that modulate rhomboid proteases. For example, in one embodiment, the atomic coordinates of TM5, or a corresponding helix in another rhomboid protease, may be used to identify compounds that inhibit the flexibility of TM5 and thereby modulate the activity of the rhomboid protease. In another embodiment, the atomic coordinates of at least a portion of a helix surrounding TM5 may be used to identify compounds in methods of the invention. In still another embodiment, the atomic coordinates of at least a portion of L1 may be used to identify compounds that may bind to and modulate the activity of the rhomboid protease.

In some embodiments, structure based drug design may be directed compound design or random compound design, and in others, selecting a compound may be performed in conjunction with computer modeling. In certain embodiments, the compound may be tested by contacting the rhomboid protease and detecting binding of the rhomboid protease and the compound, for example, by using a cell-free assay or a cell-culture assay. In such embodiments, the compound may concurrently be tested for binding and modulation of protease activity or testing for binding and modulation of rhomboid protease activity may be tested separately.

Combinatorial library technology provides an efficient way of testing a potentially vast number of different substances for their ability to modulate the activity of a rhomboid protease. In some embodiments, test substances may be screened for their ability to interact with the rhomboid protease in an enzymatic assay. For example, in one such enzymatic assay an artificial protein substrate, such as, for example, a membrane: associated protein, such as, CED-4, or C100-Spitz-Flag may be contacted with a rhomboid protease, such as GlpG. Cleavage products may then be analyzed by, for example, sodium dodecyl-sulfate polyacrylamide gel electrophoresis (SDS-PAGE). A test substance may be added to such an enzymatic, assay as a separate component, and modulation of the enzymatic activity of the rhomboid protease may be apparent by a reduction or enhancement of the amount or type of cleavage product. The modulation of activity may be indicative of binding of the test substance to the rhomboid protease.

A class of modulator compounds may be derived from a rhomboid polypeptide and/or a rhomboid substrate transmembrane domain (TMD). For example, in some embodiments, a membrane permeable peptide fragment of from about 5 to about 40 amino acids or, in certain embodiments, from about 6 to about 10 amino acids, may be prepared and utilized to modulate the activity of a rhomboid protease. In such embodiments, the peptide or peptide fragment may be modified such that it is no longer capable of being cleaved by proteolysis catalyzed by the rhomboid protease. Examples of peptide fragments that may modulate the activity of a rhomboid protease include, but not are limited to residues 141 to 144 (Ala-Ser-Gly-Ala, SEQ. ID No. 7), residues 140-144 (11e-Ala-Ser-Gly-Ala, SEQ. ID No. 8), or residues 138-144 (Ala-Ser-Ile-Ala-Gly-Ala, SEQ. ED No. 9) of the Spitz protein, or the equivalent regions of other rhomboid ligands, Ala-Gly-Ala-Ile-Ala-Gly-Gly (SEQ ID No. 1), Ala-Ile-Ala-Gly-Gly Val-Ile (SEQ ID No. 2), Ala-Ile-Ala-Gly-Gly Val-Val (SEQ ID No. 3), Tyr-Tyr-Ala-Gly-Ala-Gly Val (SEQ ID No. 4), Ala-Gly-Ala-Ile-Ala-Gly-Gly (SEQ ID No. 5), and Ala-Gly-Ala-Ile-Ala-Gly-Gly-Val-Ile-Gly-Gly (SEQ ID No. 6). Embodiments of the invention are, therefore, directed to these peptides and therapeutic compositions including peptides of sequence from SEQ ID Nos. 1-6, or a mimetic of a sequence from SEQ ID NOs. 1-6.

A variety of techniques are available for constructing peptidomimetics with the same or similar desired biological activity as the corresponding native, but with more favorable activity than the peptide with respect to solubility, stability, and/or susceptibility to hydrolysis or proteolysis (Morgan et al. (1989) Ann Rep Med Chem 24:243-252). Certain peptidomimetic compounds are based upon the amino acid sequence of the peptides of the disclosure. Often, peptidomimetic compounds are synthetic compounds having a three dimensional structure (i.e. a “peptide” motif) based upon the three dimensional structure of a selected peptide. The peptide motif provides the peptidomimetic compound with the desired biological activity, i.e. binding to GlpG or other members of the rhomboid family, wherein the binding activity of the mimetic compound is not substantially reduced, and is often the same as or greater than the activity of the native peptide on which the mimetic was modeled. Peptidomimetic compounds can have additional characteristics that enhance their therapeutic application, such as increased cell permeability, greater affinity and/or avidity and prolonged biological half-life.

Peptidomimetic design strategies are available in the art (Ripka et al. (1998) Curr Opin Chem Biol 2:441-452; Hruby et al. (1997) Curr Opin Chem Biol 1:114-119; Hruby et al. (2000) Curr Med Chem 9:945-970). One class of peptidomimetic mimics a backbone that is partially or completely non-peptide, but mimics the peptide backbone atom-for-atom and comprises side groups that likewise mimic the functionality of the side groups of the native amino acid residues. Several types of chemical bonds e.g. ester, thioester, thioamide, retroamide, reduced carbonyl, dimethylene and ketomethylene bonds, are known in the art to be generally useful substitutes for peptide bonds in the construction of protease resistant peptidomimetics. Another class of peptidomimetics comprises a small non-peptide molecule that binds to another peptide or protein, but which is not necessarily a structural mimetic of the native peptide.

Yet another class of peptidomimetics has arisen from combinatorial chemistry and the generation of massive chemical libraries. These generally comprise novel templates which, though structurally unrelated to the native peptide, possess necessary functional groups positioned on a non-peptide scaffold to serve as “topographical” mimetics of the original peptide (Ripka et al. (1998) supra).

In various embodiments, compounds that have been shown to modulate rhomboid activity, such as, DCI, TPCK, or C8E4, may be used as lead compounds in the rational drug design. These compounds may mimic the natural peptide or protein substrate binding to rhomboid proteases. Therefore, structural data from GlpG-C8E4 or DCI-bound GlpG-C8E4 represent an excellent starting point for the rational design of specific modulators of rhomboid activity. In such embodiments, the structure of the modulating compound either alone or in complex with a rhomboid protease may be used to develop mimetics that mimic this structure using, for example, structure based drug design to provide potential inhibitor compounds with particular molecular shape, size and charge characteristics.

The invention described herein also encompasses methods for identifying modulators of rhomboid protease activity. Such methods are well known in the art and may include testing libraries of randomly selected peptides or mimetics, testing libraries of known peptides or mimetics using computer methods, or designing peptides or mimetic de novo. In general such methods include analyzing known modulators, target proteins, and/or combined modulators and target proteins, and identifying elements of the modulator or the protein target important for activity, such as, for example, size, shape, density, and charge of the modulator or an active site of the target protein. The modulator may than be modified to improve an interaction with the protein by, for example, systematically varying the amino acid residues peptide modulator to effect better binding, or alternatively, the atomic coordinates of a substrate peptide or inhibitor bound to the protein may be used as a starting model which may allow for structures of mimetics to be modeled according to their physical properties, such as, but not limited to, stereochemistry, bonding, size, and charge. In such embodiments, computational analysis, similarity mapping, wherein models of the charge and/or volume of a mimetic, rather than the bonding between atoms are used for modeling and any other technique known in the art can be used in this modeling process. In a particular embodiment, the essential catalytic residues rhomboid proteases described herein may be used to develop mimetics. For example, the atomic coordinates of S201 and/or H254 of E. coli GlpG and/or substrate amino acids required for cleavage by rhomboid proteases, such as, A138, S139, I140, A141, S142, G143 and A144 of Spitz or their equivalent in other rhomboid ligands may be used for modeling.

In some embodiments, computational analysis is used to develop a template molecule onto which chemical groups which mimic the substrate may be grafted. For example, in such embodiment, a template molecule may be selected that mimics the overall shape of the substrate or inhibitor molecule, and chemical groups may be grafted onto the template molecule to complement, for example, the shape and/or charge of the active site of the target protein. The template molecule and the chemical groups grafted on to it may conveniently be selected so that the mimetic is easy to synthesize, is likely to be pharmacologically acceptable, and does not degrade in vitro while retaining the biological activity of the lead compound.

For example, FIG. 8D shows a structural model of a substrate peptide bound to the active site of GlpG that was deduced from the crystallographic data derived from crystals of GlpG-C8E4 soaked in MIC2. In this model, computational analysis of the electron density associated with the substrate peptide was performed and a peptide backbone was constructed that appeared to correspond to the electron density observed in crystallographic data. The side chains associated with the amino acid sequence of MIC2 were then grafted onto peptide backbone such that each amino acid side chain fit into the active site of GlpG. For example, the putative amino acid side chains of the substrate peptide were positioned such that the side chains fit within the observed electron density in the crystallographic data and the electron density of the amino acids that make up the active site binding cleft of GlpG and the electron density associated with the putative side chain did not overlap. An rmsd for the modeled substrate peptide was then performed to ensure accuracy.

Such analysis can be carried out to produce any number of putative substrate-like inhibitors by, for example, designing a non-peptide organic molecule inhibitor that fits within the observed electron density of the putative substrate or designing a peptide like compound that is resistant to proteolysis and fits within the observed electron density. Moreover, similar analysis can be carried out using the electron density associated with rhomboid inhibitors, such as, for example, C8E4 or DCI. In performing such analysis putative molecular interactions between the inhibitor and the rhomboid protease may be improved by, for example, designing a mimetic with additional hydrogen bonding or van der Waals contacts.

Methods for performing structural comparisons of atomic coordinates of molecules including those derived from protein crystallography are well known in the art, and any such method may be used in various embodiments to test candidate rhomboid binding compounds for the ability to bind a portion of rhomboid. In such embodiments, atomic coordinates of designed, random or stored candidate compounds may be compared against a portion of the rhomboid structure or the atomic coordinates of a compound bound to rhomboid. In other such embodiments, a designed, random or stored candidate compound may be brought into contact with a surface of the rhomboid, and simulated hydrogen bonding and/or van der Waals interactions may be used to evaluate or test the ability of the candidate compound to bind the surface of the rhomboid. Structural comparisons, such as those described in the preceding embodiments may be carried out using any method, such as, for example, a distance alignment matrix (DALI), Sequential Structure Alignment Program (SSAP), combinatorial extension (CE) or any such structural comparison algorithm. Compounds that appear to mimic a portion of the rhomboid structure under study or a compound known to bind a rhomboid, such as, for example, a substrate protein, or that are substantially complementary and have a likelihood of forming sufficient interactions to bind to, rhomboid may be identified as a potential rhomboid binding compound.

In some embodiments, compounds identified as described above may conform to a set of predetermined variables. For example, in one embodiment, the atomic coordinates of an identified rhomboid binding compound when compared with a native rhomboid binding compound or a portion of the rhomboid protease using one or more of the above structural comparison methods may deviate from an rmsd of less than about 5 Å. In another embodiment, the atomic coordinates of the compound may deviate from the atomic coordinates of rhomboid by less than about 2 Å. In still another embodiment, the identified rhomboid binding compound may include one or more specific structural features known to exist in a native rhomboid binding compound or a portion of the rhomboid protease, such as, for example, a surface area, shape, charge distribution over the entire compound or a portion of the identified compound.

Various embodiments of the invention also include a system for identifying a rhomboid protease modulator. Such systems may include a processor and a computer readable medium in contact with the processor. The computer readable medium of such embodiments may at least contain the atomic coordinates of a rhomboid protease. In some embodiments, the computer readable medium may further contain one or more programming instructions for comparing at least a portion of the atomic coordinates of the rhomboid protease with atomic coordinates of candidate compounds included in a library of compounds. In other embodiments, the computer readable medium may further contain one or more programming instructions for designing a compound that mimics at least a portion of the rhomboid protease or that is substantially complementary to a portion of the rhomboid protease. In still other embodiments, the computer readable medium may contain one or more programming instructions for identifying candidate compounds or designing a compound that mimics a portion of rhomboid protease within one or more user defined parameters. For example, in some embodiments, a compound may include a charged molecule at a particular position corresponding to one or more positions within the atomic coordinates of rhomboid protease, and in other embodiments, the compound may deviate from the carbon backbone or surface model, representation of rhomboid protease by, for example, an rmsd of less than about 5 Å, and in certain embodiments, the rmsd may be less than about 2 Å. In still other embodiments, a user may determine the size of a candidate compound or the portion of the rhomboid protease that is utilized in identifying mimetic candidate compounds. Further embodiments may include one or more programming instructions for simulating binding of an identified candidate compound to rhomboid protease or a portion of the rhomboid protease. Such embodiments may be carried out using any method known in the art, and may provide an additional in silico method for testing identified candidate compounds.

Compounds identified by the various methods embodied herein may be synthesized by any method known in the art. For example, identified compounds may be synthesized using manual techniques or by automation using in vitro methods such as, various solid state or liquid state synthesis methods. Direct peptide synthesis using solid-phase techniques is well known and utilized in the art (see, e.g., Stewart et al., Solid-Phase Peptide Synthesis, W. H. Freeman Co., San Francisco, Calif. (1969); Merrifield, J. Am. Chem. Soc., 85:2149-2154 (1963)). Automated synthesis may be accomplished, for example, using a Peptide Synthesizer using manufacturer's instructions. Additionally, in some embodiments, one or more portion of the rhomboid modulators described herein may be synthesized separately and combined using chemical or enzymatic methods to produce a full length modulator.

In another embodiment, rhomboid protease binding peptides may be modified by replacement of ones or more naturally occurring side chains of the 20 genetically encoded amino acids (or D amino acids) with other side chains to produce peptide mimetics. For example, the other side chains may contain groups such as alkyl, lower alkyl, cyclic 4-, 5-, 6-, to 7-membered alkyl, amide, amide lower alkyl, amide di(lower alkyl), lower alkoxy, hydroxyl, carboxy and the lower ester derivatives thereof, and with 4-, 57-, 6-, to 7-membered heterocyclics. For example, proline analogs can be made in which the ring size of the proline residue is changed from 5 members to 4, 6 or 7 members. Cyclic groups can be saturated or unsaturated, and if unsaturated, can be aromatic or non-aromatic. Heterocyclic groups can contain one or more nitrogen, oxygen, and/or sulfur heteroatoms. Examples of such groups include furazanyl, furyl, imidazolidinyl, imidazolyl, imidazolinyl, isothiazolyl, isoazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. I-pyrrolidinyl), pyrrolinyl, pyrrolyl, thiadiazolyl, thiazolyl, thienyl, thiomorpholinyl, (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidomimetics may also have amino acid residues that have been chemically modified by phosphorylation, sulfonation, biotinylation, or the addition or removal of other moieties.

In various embodiments, peptides or peptide fragments, such as those described above, may be modified to inhibit proteolysis of peptide or peptide fragment and/or to improve stability of the peptide or peptide fragment. For example, in some embodiments, the peptide or peptide fragments may be modified by C terminal addition of a transition state analogues for serine, cysteine and threonine proteases, such as, but not limited to, chloromethyl ketone, aldehyde, boronic acid, and the like. In other embodiments, the N-terminus of a peptide fragment may be blocked with carbobenzyl. Other examples of methods for stabilizing peptides or peptide fragments are well known in the art and can be found, for example, in Proteolytic Enzymes 2nd Ed, Edited by R. Beynon and J. Bond Oxford University Press 2001.

Modulators identified by any method provided herein may further undergo screening to ensure that the compound functions as a modulator. For example, compounds which model the three-dimensional conformation of a rhomboid ligand such as Spitz may be screened to ensure binding to a rhomboid protease and inhibition of proteolysis using, for example, in vitro methods described hereinabove. Following confirmation that the mimetic binds to and inhibits rhomboid activity, the mimetic may be investigated further by, for example, examining the ability of the mimetic to modulate rhomboid-mediated cellular activities in, vivo.

In an embodiment, modulators of rhomboid protease activity identified as described herein may be used as a therapeutic for the treatment of diseases such as cancer or neurodegenerative diseases. Accordingly, an embodiment of the disclosure comprises administering to a cell a therapeutically effective amount of the compounds to stimulate or inhibit the activity of one or more rhomboid protease. In such embodiments, the cell may be contained within a tissue, and the tissue may be located in a living organism, such as, on an animal, or a mammal, and in some cases a human.

Modulators of various embodiments of the invention may be manufactured or used in formulations of compositions such as medicaments, pharmaceutical compositions or drugs, and such formulations may be administered to individuals for the treatment of disorders as described below. Methods of the invention may, thus, include formulating mimetics in a pharmaceutical composition with a pharmaceutically acceptable excipient, vehicle or carrier for therapeutic application. For example, a method of making a pharmaceutical composition may include identifying a modulator of rhomboid activity using a method described hereinabove, synthesizing, preparing and/or isolating the modulator, admixing the modulator with a pharmaceutically acceptable excipient, vehicle or carrier, and, optionally, other ingredients to formulate pharmaceutical compositions.

Pharmaceutical compositions may include modulators of rhomboid proteases that have been additionally modified to, for example, optimize activity, increase half-life, or reduce side effects of the pharmaceutical composition upon administration to an individual. Modification of pharmacologically active compounds to improve pharmaceutical properties is a known approach to the development of pharmaceuticals based on a “lead” compound. This might be desirable where the active compound is difficult or expensive to synthesize or where it is unsuitable for a particular method of administration. The design, synthesis and testing of modified active compounds, including mimetics, may be used to avoid randomly screening large number of molecules for a target property. For example, TPCK and DCI inhibit rhomboid activity, but these compounds lack specificity and may produce undesirable side-effects if used therapeutically. However, these compounds may be used as “lead” compounds for the development of rhomboid inhibitors with improved specificity.

A pharmaceutical composition comprising a rhomboid modulator as described herein, may be administered to an individual for the treatment or preventative treatment of a pathogenic infection or a condition associated with or mediated by rhomboid activity, such as, for example cardiovascular disorders, including disorders associated with blood coagulation, inflammatory disorders, cancer and the like.

Examples of cardiovascular disorders that may be treated using mimetics of the invention include, but not be, limited to, cardiac myxoma, acute myocardial infarction, stroke, in particular hemorrhagic stroke, ischaemic (coronary) heart disease; atherosclerosis, myocardial ischaemia (angina) and disorders associated with blood coagulation such as cerebral thrombosis, cerebral embolism coronary artery thrombolysis, arterial and pulmonary thrombosis and embolism, and various vascular disorders such as peripheral arterial obstruction, deep vein thrombosis, disseminated intravascular coagulation syndrome, thrombus formation after artificial blood vessel operation or after artificial valve replacement, re-occlusion and re-stricture after coronary artery by-pass operation, re-occlusion and re-stricture after PTCA (percutaneous transluminal coronary angioplasty) or PTCR (percutaneous transluminal coronary re-canalization) operation and thrombus formation at the time of extracorporeal circulation.

Examples of inflammatory disorders that may be treated using mimetics of the invention include, but are not be limited to, allergy, asthma, atopic dermatitis, Crohn's disease, Felty's syndrome, gingivitis, pelvic inflammatory disease, periodontitis, polymyositis/dermatomyositis, psoriasis, rheumatic fever, rheumatoid arthritis, skin inflammatory diseases, spondylitis, systemic lupus erythematosus, ulcerative colitis, uveitis, vasculitis and inflammation caused by sepsis or ischaemia.

Examples of cancer or cancerous conditions that may be treated using mimetics of the invention include, but are not limited to, histocytoma, glioma, glioblastoma, astrocyoma, osteoma, lung cancer, small cell lung cancer, gastrointestinal cancer, bowel cancer, oral cancer, colon cancer, breast cancer, oesophageal cancer, ovarian carcinoma, prostate cancer, testicular cancer, liver cancer, kidney cancer, bladder cancer, pancreas cancer, skin cancer and brain cancer.

Other disorders mediated by rhomboid activity include diabetes, disorders of peripheral nervous system, pneumonia, adult respiratory distress syndrome, chronic renal failure and acute hepatic failure.

The invention described herein encompasses pharmaceutical compositions including a therapeutically effective amount of an inhibitor in dosage form and a pharmaceutically acceptable carrier, wherein the compound inhibits the activity of one or more rhomboid protease. In another embodiment, such compositions include a therapeutically effective amount of an inhibitor in dosage form and a pharmaceutically acceptable carrier in combination with a chemotherapeutic and/or radiotherapy, wherein the inhibitor inhibits the activity of one or more rhomboid protease, promoting apoptosis and enhancing the effectiveness of the chemotherapeutic and/or radiotherapy. In various embodiments of the invention, a therapeutic composition for modulating the activity of one or more rhomboid protease can be a therapeutically effective amount of a rhomboid inhibitor.

The compounds of the present invention can be administered in the conventional manner by any route where they are active. For example, administration can be, but is not limited to, systemic, parenteral, subcutaneous, intravenous, intramuscular, intraperitoneal, topical, transdermal, oral, buccal, or ocular routes, or intravaginally, by inhalation, by depot injections, or by implants. Thus, modes of administration for the compounds of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the compounds of the present invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker; Inc. (1979); and Goodman & Gilman's The Pharmaceutical Basis of Therapeutics, 6th Edition, MacMillan Publishing Co., New York (1980) can be consulted.

The compounds of the present invention can be formulated for parenteral administration by injection, e.g., by bolus injection or continuous infusion. The compounds can be administered by continuous infusion subcutaneously over a period of about 15 minutes to about 24 hours. Formulations for injection can be presented in unit dosage form, e.g., in ampoules or in multi-dose containers, with an added preservatives. The compositions can take such forms as suspensions, solutions or emulsions in oily or aqueous vehicles, and can contain formulatory agents such as suspending, stabilizing and/or dispersing agents.

For oral administration, the compounds can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds of the present invention can also be formulated in rectal compositions such as suppositories or retention enemas, e.g., containing conventional suppository bases such as cocoa butter or other glycerides.

In addition to the formulations described previously, the compounds of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the present invention, for example, can be applied to a plaster, or can be applied by transdermal, therapeutic systems that are consequently supplied to the organism.

Pharmaceutical compositions of the compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compounds of the present invention can also be administered in combination with other active ingredients, such as, for example, adjuvants, protease inhibitors, or other compatible drugs or compounds where such combination is seen to be desirable or advantageous in achieving the desired effects of the methods described herein.

This invention and embodiments illustrating the method and materials used may be further understood by reference to the following non-limiting examples.

### EXAMPLES

### Protein Preparation

The transmembrane core domain of GlpG (residues 87-276) was purified as described previously (See Wu et al. (2006) Nature Struct Mol Biol. 13:1084-1091, hereby incorporated by reference in its entirety).

### Crystallization and Data Collection

Crystals of GlpG were grown at 22° C. using the hanging drop vapor diffusion method. The well buffer, which contains 0.1 M Tricine (pH 7.4), 6% (w/v) PEG3000 and 50-100 mM Li2SO4, was identical to that reported by Wu et al. All crystals discussed in this manuscript were grown in the presence of 0.5% C8E4, which was added to the protein solutions right before setting up trays. To: derivatize crystals with DCI, single crystals were transferred to well buffer plus 5-10 mM freshly prepared DCI (Calbiochem) and 20% Glycerol (v/v) and harvested for freezing after 5-15 minutes. To obtain crystals soaked with substrate peptides, well buffer plus 2-5 mM peptides and 20% glycerol (v/v) was pre-cooled to −12° C. in a cold nitrogen stream. Then single crystals were transferred to the pre-cooled solution and harvested for freezing after 5-15 minutes. The crystals belong to the space group P31 and contain two molecules per asymmetric unit. The unit cell dimensions are similar to those reported by Wu et al. Crystals were equilibrated in a cryoprotectant buffer containing reservoir buffer plus 0.5% NG and 20% glycerol (v/v) and were flash frozen in a cold nitrogen stream at −170° C. The native data set was collected at NSLS beamline X29 and processed using the software Denzo and Scalepack.

### Structure Determination

Initial phases for the three structures were obtained from the PDB entry 2NRF by molecular replacement implemented in MolRep. Models were built using 0 and refined using Refmac5. Tight non-crystallographic symmetry (NCS) was applied to the two molecules of one asymmetric unit during early refinement. Flexible residues 238-251 were rebuilt based on their corresponding Sigma A weighted omit densities. After the completion of the models, TLS groups were introduced to model the data anisotropy. During late-stage refinement, NCS was loosened and released. In DCI-bound structure, DCI was modeled based on a DCI-inhibited seine protease. DCI was covalently-linked to Ser201 hydroxyl group with a bond distance restrained at 1.3 Å. The final atomic models contain residues 92-272 for C8E4-bound and substrate-soaked structures, and 93-272 for DCI-bound structure. No residues are in the disallowed region of the Ramachandran plot.

### Protease Activity Assay

The proteolytic activity of GlpG was examined as described by Wu et al.

What is claimed is:

1. A method for preparing a rhomboid protease modulating compound comprising:
applying a three-dimensional molecular modeling-algorithm to the atomic coordinates of at least a portion, of rhomboid protease;
determining spatial coordinates of at least a portion of rhomboid protease;
electronically screening stored spatial coordinates of candidate compounds against the spatial coordinates of at least a portion of rhomboid protease;
identifying a compound that is substantially similar to at least a portion of rhomboid protease; and
synthesizing the identified compound.

2. The method of claim 1, further comprising identifying a candidate compound that deviates from the atomic coordinates of at least a portion of rhomboid protease by a root mean square deviation of less than about 5 angstroms.

3. The method of claim 1, further comprising testing the identified compound for binding at least a portion of rhomboid protease.

4. The method of claim 1, further comprising testing the identified compound for inhibiting rhomboid protease activity.

5. The method of claim 1, wherein the step of electronically screening stored spatial coordinates further comprises identifying a compound that has a shape, a charge distribution, a size or a combination thereof substantially similar to a portion of rhomboid protease.

6. The method of claim 1, wherein at least a portion of the rhomboid protease comprises at least a portion of one or more of: transmembrane helix 5 (TM5); transmembrane helix 4 (TM4); transmembrane helix 6 (TM6); loop 5 (L5); or loop 1 (L1).

7. The method of claim 6, wherein the identified compound inhibits entry of a substrate protein into an active site of the rhomboid protease.

8. The method of claim 6, wherein the identified compound enhances entry of a substrate protein into an active site of the rhomboid protease.

9. A method for preparing a rhomboid protease inhibitor comprising:
applying a three-dimensional molecular modeling algorithm to atomic coordinates of a rhomboid protease having a bound substrate peptide; or
applying a three-dimensional molecular modeling algorithm to atomic coordinates of a rhomboid protease having a rhomboid binding compound;
determining spatial coordinates of at least a portion of substrate peptide or binding compound;
electronically screening stored spatial coordinates of candidate compounds against the spatial coordinates of at least a portion of substrate peptide or binding compound;
identifying a compound that is substantially complementary to the substrate peptide or binding compound; and
synthesizing the identified compound.

10. The method of claim 9, further comprising identifying a compound that has a shape, a charge distribution, a size or a combination thereof substantially similar to at least a portion of the substrate peptide or binding compound.

11. The method of claim 9, wherein the substrate peptide is Spitz, C100-Spitz-Flag, a peptide derived form Toxoplasma gondii micronemal proteins, MIC2, or a combination thereof.

12. The method of claim 9, wherein the identified compound inhibits entry of substrate into an active site of the rhomboid protease.

13. The method of claim 9, further-comprising:
identifying one or more substrate peptides;
isolating at least a portion of the one or more substrate peptides where the rhomboid protease is likely to bind the one or more substrate peptides;
determining spatial coordinates of at least a portion of the one or more substrate peptides; and
identifying a compound that is substantially similar to at least a portion of the one or more substrate peptides.

14. The method of claim 13, wherein the step of isolating one or more substrate peptides further comprises:
identifying more than one substrate peptides;
performing an alignment of the more than one substrate peptides; and
isolating at least a portion of the more than one substrate peptides that share sequence similarity or secondary structure similarity.

15. The method of claim 9, further comprising testing the identified compound for binding to the rhomboid-protease.

16. The method of claim 9, wherein the synthesized compound comprises one or more modified peptide bond.

17. The method of claim 9, wherein the synthesized compound is modified such that rhomboid protease mediated proteolysis of the synthesized compound cannot occur.

18. The method of claim 9, wherein the rhomboid binding compound comprises one or more of: tetraethyleneglycol monoctyl ether (C8E4), dichloroisocourmarin (DCI) or a combination thereof

19. A pharmaceutical composition comprising:
an effective amount of a compound having a three-dimensional structure corresponding to atomic coordinates of at least a portion of a rhomboid protease, a substrate peptide of a rhomboid protease or a rhomboid protease binding compound; and
a pharmaceutically acceptable excipient or carrier.

20. The pharmaceutical composition of claim 19, wherein the compound binds to the rhomboid protease.

21. A system for identifying rhomboid protease modulators comprising:
a processor; and
a processor readable storage medium in communication with the processor readable storage medium comprising the atomic coordinates of at least a portion of rhomboid protease.

22. The system of claim 21, wherein the processor readable storage medium further comprises one or more programming instructions for:
applying a three-dimensional modeling algorithm to the atomic coordinates of the rhomboid protease;
determining spatial coordinates of at least a portion of the rhomboid protease;
electronically screening spatial coordinates of candidate compounds with the spatial coordinates of at least a portion of the rhomboid protease; and
identifying a candidate compound whose spatial coordinates are substantially similar to the spatial coordinates of at least a portion of the rhomboid protease; or
identifying a candidate compound whose spatial coordinates are substantially complementary to the spatial coordinates of at least a portion of the rhomboid protease.

23. The system of claim 22, wherein the one or more programming instructions for identifying a candidate compound whose spatial coordinates are substantially similar to the spatial coordinates of at least a portion of the rhomboid protease comprise one or more programming instructions for identifying a compound that deviates from the spatial coordinates of at least a portion of the rhomboid protease by a user defined threshold.

24. The system of claim 22, wherein the one or more programming instructions for identifying a compound whose spatial coordinates are substantially similar to at least a portion of the rhomboid protease comprise one or more programming instructions for identifying a compound having one or more of:
a size within a user defined threshold;
a charge within a user defined threshold; or
a shape with a user defined threshold.

25. The system of claim 22, wherein the one or more programming instructions for electronically screening spatial coordinates of a candidate compound comprises one or more programming instructions for simulating binding of the candidate compound to the rhomboid protease.

26. The system of claim 21, further comprising an output device in communication with the processor.

27. The system of claim 26, wherein the processor readable storage medium further comprises one or more programming instructions for:
applying a three-dimensional modeling algorithm to the atomic coordinates of rhomboid protease;
determining spatial coordinates of at least a portion of the rhomboid protease;
generating a visual signal and relaying the visual signal to the output device; and
electronically designing a compound that is substantially similar to at least a portion of the rhomboid protease; or
electronically designing a compound that is substantially complementary to at least a portion of the rhomboid protease.

28. A rhomboid protease binding compound comprising a molecule having a three-dimensional structure corresponding to atomic coordinates derived from at least a portion of an atomic model of a rhomboid protease, a rhomboid protease bound to an inhibitor or a rhomboid protease bound to a substrate.

29. The compound of claim 28, wherein the inhibitor is tetraethyleneglycol monoctyl ether (C8E4), dichloroisocourmarin (DCI) or a combination thereof.

30. The compound of claim 28, wherein the substrate is Spitz, C100-Spitz-Flag, a peptide derived form Toxoplasma gondii micronemal proteins, or a combination thereof.

31. The compound of claim 28, wherein the molecule has a three-dimensional structure corresponding to atomic coordinates of at least a portion of tetraethyleneglycol monoctyl ether (C8E4), dichloroisocourmarin, (DCI), Spitz, C100-Spitz-Flag, a peptide derived form Toxoplasma gondii micronemal proteins, or a combination thereof.

32. The compound of claim 28, wherein the molecule has a shape, a charge, a size or combinations thereof substantially corresponding to a portion of a rhomboid protease.

33. The compound of claim 28, wherein the molecule binds at an interface between transmembrane helix 5 (TM5) or its structural equivalent in another rhomboid protease and another structural feature of the rhomboid protease.

34. The compound of claim 28, wherein the molecule has a shape, a charge, a size or combinations thereof substantially complementary to a portion of a rhomboid protease.

35. The compound of claim 34, wherein the molecule inhibits access of substrate to the active site of the rhomboid protease.

36. The compound of claim 28, wherein the molecule binds to at least a portion of the rhomboid protease with a: greater affinity than a naturally occurring substrate.

37. The compound of claim 28, wherein the molecule inhibits rhomboid protease mediated proteolysis.

38. The compound of claim 28, further comprising a pharmaceutically acceptable excipient or carrier.

39. The compound of claim 28, wherein the molecule deviates from the atomic coordinates of at least a portion of the rhomboid protease by a root mean square deviation of less than about 10 angstroms.

40. The compound of claim 28, wherein the molecule deviates from the atomic coordinates of at least a portion of the rhomboid protease by a root mean square deviation of less than about 2 angstroms.

41. The compound of claim 28, wherein the molecule is a peptide or peptidomimetic of sequence selected from:
(SEQ ID No. 1)
Ala-Gly-Ala-Ile-Ala-Gly-Gly,
(SEQ ID No. 2)
Ala-Ile-Ala-Gly-Gly-Val-Ile,
(SEQ ID No. 3)
Ala-Ile-Ala-Gly-Gly-Val-Val,
(SEQ ID No. 4)
Tyr-Tyr-Ala-Gly-Ala-Gly-Val,
(SEQ ID No. 5)
Ala-Gly-Ala-Ile-Ala-Gly-Gly,
(SEQ ID No. 6)
Ala-Gly-Ala-Ile-Ala-Gly-Gly-Val-Ile-Gly-Gly,
(SEQ. ID No. 7)
Ala-Ser-Gly-Ala,
(SEQ. ID No. 8)
Ile-Ala-Ser-Gly-Ala,
and
(SEQ. ID No. 9)
Ala-Ser-Ile-Ala-Gly-Ala.
